(19) United States
(12) Patent Application Publication
(10) Pub. No.: US 2011/0160647 A1
(43) Pub. Date: Jun. 30, 2011

(54) Angioplasty Balloon Catheter with Active Delivery Medicament(s) Using Ultrasonic Means

(76) Inventors: Ross Tsukashima, San Diego, CA (US); Don Shin, Poway, CA (US); Eugene Jung, Beechwood, OH (US); Michael Klicpera, La Jolla, CA (US)

(21) Appl. No.: 12/982,834

(22) Filed: Dec. 30, 2010

Related U.S. Application Data
(60) Provisional application No. 61/291,348, filed on Dec. 30, 2009.

(51) Int. Cl. A61B 17/20

(52) U.S. Cl. 604/22

(57) ABSTRACT

The present disclosure relates to active drug eluting angioplasty balloon which utilizes ultrasonic energy to facilitate the release of the bioactive drug thereby avoids many of the drawbacks of prior art drug eluting devices.

### RELATED APPLICATIONS

This application claims priority under 35 U.S.C. Section 119(e) to U.S. Provisional Application Ser. No. 61/291,348 filed on Dec. 30, 2009.

### FIELD OF THE INVENTION

The present disclosure relates to active drug eluting balloon apparatus useful for medical treatments. More specifically, the active medicament eluting balloon includes an ultrasonic means to facilitate the active release of the medicament from the balloon and into the treated vessel wall.

### BACKGROUND OF THE INVENTION

The first procedure to treat blocked coronary arteries was coronary artery bypass graft surgery (CABG), wherein a section of vein or artery from elsewhere in the body is used to bypass the diseased segment of coronary artery. In 1977, Andreas Grüntzig introduced percutaneous transluminal coronary angioplasty (PTCA), also called balloon angioplasty, in which a catheter was introduced through a peripheral artery and a balloon expanded to dilate the narrowed segment of artery.

As equipment and techniques improved, the use of PTCA rapidly increased, and by the mid-1980s, PTCA and CABG were being performed at equivalent rates. Balloon angioplasty was generally effective and safe, but restenosis was frequent, occurring in ˜30-40% of cases, usually, within the first year after dilation. In ˜3% of balloon angioplasty cases, failure of the dilation and acute or threatened closure of the coronary artery (often because of dissection) prompted emergency CABG.

Dotter and Melvin Judkins had suggested using prosthetic devices inside arteries (in the leg) to maintain blood flow after dilation as early as 1964. In 1986, Fuel and Sigwart implanted the first coronary stent in a human patient. Several trials in the 1990s showed the superiority of stent placement over balloon angioplasty. Restenosis was reduced because the stent acted as a scaffold to hold open the dilated segment of artery; acute closure of the coronary artery (and the requirement for emergency CABG) was reduced, because the stent precluded acute closure allowing repaired dissections of the arterial wall to heal. By 1999, stents were used in 84% of percutaneous coronary interventions (i.e., those done via a catheter, and not by open-chest surgery.)

Early difficulties with coronary stents included a risk of early thrombosis (clotting) resulting in occlusion of the stent. Coating stainless steel stents with other substances such as platinum or gold were evaluated but, ultimately, did not eliminate this problem. High-pressure balloon expansion of the stent to ensure its full apposition to the arterial wall, combined with drug-therapy using aspirin and another inhibitor of platelet aggregation (usually ticlopidine or clopidogrel) nearly eliminated this risk of early stent thrombosis.

Though it occurred less frequently than with balloon angioplasty or other techniques, stents nonetheless remained vulnerable to restenosis, caused almost exclusively by neointimal tissue growth. To address this issue, developers of drug-eluting stents used the devices themselves as a tool for delivering medication directly to the arterial wall. While initial efforts were unsuccessful, it was shown in 2001 that the release (elution) of drugs with certain specific physicochemical properties from the stent can achieve high concentrations of the drug locally, directly at the target lesion, with minimal systemic side effects ^{[8]}. As currently used in clinical practice, “drug-eluting” stents refers to metal stents which elute a drug designed to limit the growth of neointimal scar tissue, thus reducing the likelihood of stent restenosis.

Many prior deployable medical devices have used bioactive medicaments with polymeric and other coatings/binding agents on the surfaces of deployable devices so that the bioactive medicaments (e.g. Paclitaxel, Sirolimus (Rapamycin), Tacrolimus, Everolimus and Zotarolimus) will be passively released over time after the medical device has been deployed. Polymeric coatings can hold bioactive medicaments onto the surface of deployable medical devices during deployment of the medical device within a treatment segment of an artery and passively released over time. This occurs because the polymeric coating degrades over time allowing the bioactive medicament to diffuse into the bloodstream or tissue. However uses of passive polymeric coatings are not without problems.

Drug eluting stents that have bioactive medicaments with or without a polymeric coating have been known to have long term chronic complications. This might be due to the long term release of the medicament and/or due to the fact that the stent is an irritant implant within the tissue. It has been hypothesized that the medicaments precludes endothelialization of the stent which triggers thrombus formation. Recent reports have indicated that there may be an increased risk of late stent thrombosis with the use of drug-eluting stents, as compared with bare-metal stents.

Non-implanted percutaneous transluminal coronary angioplasty (PTCA) balloons catheters can be coated with Paclitaxel, Sirolimus (Rapamycin), Tacrolimus, Everolimus and Zotarolimus but due to the passive release need to have long inflation times. They can also have contra-indicated ischemic plots, proximal lesions/left main coronary artery and the loss of drug eluting material off the device during delivery can exhibit a high degree of washout (release of medicament into non-targeted areas, primarily non-diseased sections of artery) which can be greater than 80%. Weeping catheters/balloons can also have long inflation times with a high degree of washout which can be greater than 95%.

The polymer system used to encapsulate the medicament is of paramount importance. It must ensure that the medicament is not lost in the bloodstream (washout) during transport of the catheter through the human arterial system to the target lesion site in the coronary arteries. In devices without an active dispersing mechanism the coating must not be overly robust as to preclude drug delivery at the intended target site. This requires a fine balance where the coating is sufficiently robust to avoid drug washout during transit but is not overly robust so that the drug can be delivered at the intended targeted site. This need to balance the properties of the polymer coating remains problematic and, to date, results from drug eluting balloons have been disappointing in clinical testing.

Ultrasound energy is a configurable modality that can create a variety of different bioeffects in tissue. Low frequency, below 1 MHz, pulsed ultrasound tends to produce mechanical impacts such as cell lyses, fractionation, cavitation, and microstream formation. High frequency continuous power delivery tended to produce thermal heating in tissue. Medical applications of therapeutic ultrasound include lithotripsy of renal stones, treatment of prostatic hyperplasia, prostate cancer and testicular tumors, ablation of uterine tumors and heart disease. In cardiology, the use of High Intensity Focused Ultrasound (HIFU) in a catheter based system was recently investigated in a FDA approved clinical trial for the treatment of atrial fibrillation.

As disclosed in (Circulation. 1997; 95:1360-1362 Paul G. Yock, MD; Peter J. Fitzgerald, MD, PhD, the potential for using therapeutic ultrasound to treat atherosclerosis and thrombosis has been appreciated for decades, but actual development efforts were slow to get under way. Catheter-based delivery systems for therapeutic ultrasound were first conceived and patented in the 1960s. Dedicated in vivo experimental work began in the early 1970s with the demonstration by Sobbe and colleagues that ultrasound delivered through a wire probe could be used to disrupt blood clots in animals. As with many other technologies in cardiology, however, it was the explosive growth of angioplasty in the 1980s that brought attention, funding, and real momentum to the development of therapeutic catheter ultrasound.

In the late 1980s, two groups, headed by Siegel and Rosenschein, began serious development efforts to address these issues. The resulting catheter designs have converged on some basic features. The current catheters from both groups are built around a solid-metal wave guide made of titanium or aluminum alloy. In the distal segment, which must be relatively flexible, the wire is either tapered or replaced by several thinner wire components. At the tip of the probe, there is a ball of larger diameter (1.2 to 1.7 mm), designed to increase energy delivery to the target. Proximal to this ball tip, the wire guide is ensheathed in plastic catheter. The catheters accept a standard guide wire in some version of a “rail” design and can be delivered through conventional guiding catheters. The proximal end of the ultrasound catheter is attached to an ultrasound transducer with a frequency of ≈20 kHz (compared with 20 to 30 MHz for intravascular ultrasound imaging transducers). The power at the transducer is 16 to 20 W, but because of loss of energy in the wave guide, the power actually delivered to the lesion is reduced by 50% or more.

These ultrasound catheters were designed to penetrate occlusions in arteries by vibrating a ball tip at the far distal end of the device against occlusive tissue. The action of the ball tip predisposing the device to puncture and cross the lesion. The effects of the ultrasound energy on normal arterial wall are now known to be to be relatively innocent under conditions simulating clinical use. Initial in vitro and animal studies raised concern about thermal effects, recording temperatures as high as 50° C. at the probe tip during continuous administration of ultrasound energy. This led to a number of strategies for temperature reduction, including saline flushing, use of pulsed instead of continuous ultrasound, and limited periods of sonication in a given treatment cycle (typically 30 or 60 seconds). With these modifications, the degree of heating has been reduced to <5° C., and histology studies have shown minimal evidence for thermal damage. One unanticipated and fascinating beneficial effect of catheter ultrasound is its ability to induce local vasodilation in the region of the probe tip. The in vitro studies of Fischell et al demonstrated ultrasound dose-dependent, endothelium-independent smooth muscle cell relaxation. These investigators suggested that ultrasound may promote a reversible disruption of the actin filament interaction in the contractile apparatus, leading to muscle cell relaxation. Initial studies of catheter-based ultrasound were performed in peripheral vessels in the late 1980s. In the first 45 patients reported by Siegel et al, 86% of completely occluded segments were recanalized using ultrasound. The ability of the ultrasound probe to induce local vasodilation (and to overcome spasm) was clearly demonstrated. There was no angiographic or clinical evidence of distal embolization. Restenosis, judged by ankle-brachial index, was 20%.

The first clinical application of therapeutic ultrasound in coronary arteries was reported by Siegel et al. in 1994. 44 procedures were clinically successful, with all but 1 using balloon angioplasty after the ultrasound treatment. In 7 of 44 cases, the ultrasound probe was not successful in crossing the lesion; however, in 9 other cases of complete occlusion, the probe was successful where conventional techniques had failed. The average residual stenosis after ultrasound treatment alone was 71%; after balloon dilation, this was further reduced to 34%. Although only 14 of the patients had completed a 6-month follow-up at the time of the report, the rate of revascularization was high enough (3 among these 14) to lead the authors to suggest that there may be “no major effect on restenosis” compared with standard catheter techniques. In the 7 patients with acute myocardial infarction, ultrasound treatment appeared to be successful in reducing thrombus burden.

Rosenschein et al. extend this experience with coronary ultrasound thrombolysis in their report on the first 15 patients in the feasibility phase of the ACUTE trial (Analysis of Coronary Ultrasound Thrombolysis Endpoints). Patients were treated in two stages: first with the ultrasound catheter and then with balloon angioplasty if required. The authors report that after they stabilized their technique in the first case, the remaining 14 applications of ultrasound were successful. Taken together, these findings suggest that the ultrasound catheter was effective in substantially reducing thrombus burden and, to an intermediate degree, relieving stenoses.

Other catheters with different mechanisms for disrupting clots are being developed. The most extensively tested technique at this point is the “hydrolysis” approach, in which water jets break up thrombus and, in one design, create suction through a Venturi effect to help remove the particulate. The ultrasound catheters do have two potential advantages of uncertain importance: (1) they tend to prevent and even overcome spasm, and (2) ultrasound can be effective in ablating plaque as well as thrombus.

Initial experience suggests that there are at least some cases in which ultrasound may be more effective than conventional techniques for crossing complete occlusions. Ultrasound does have the ability to ablate fibrocalcific tissue, which is the major source of difficulty for the interventionist in dealing with old occlusions. Another fascinating potential application for ultrasound stems from its ability to enhance the compliance of a lesion. The in vitro studies of Demer et al. indicate that ultrasound treatment produces a large increase in lesion distensibility, presumably by disrupting fibrous elements and calcium within the plaque. It follows that there may be a role for ultrasound in heavily fibrocalcific lesions, particularly in the context of stenting. Pretreatment of these segments with ultrasound might allow for full expansion of stents at relatively low pressures, potentially reducing trauma to the vessel wall.

Despite improvements to device design there remains the lack of a satisfactory solution to treat recurrent restenosis, particularly in-stent restenosis, where it is desirable to re-open the artery without having to place a second stent (or more). Multiple stents placed at the same targeted lesion fare worse so there is a need to eliminate neointimal tissue formation without using a stent platform.

Hence, there is a need for a drug eluting medical device which has an expandable member coated with a bioactive medicament that is actively released, which overcomes the shortcomings of prior art devices.

### SUMMARY OF THE INVENTION

The present disclosure relates to active drug eluting angioplasty balloon which utilizes ultrasonic energy to facilitate the release of the bioactive drug thereby avoids many of the drawbacks of prior art drug eluting devices.

The present invention is an angioplasty balloon catheter which is coated with a bioactive medicament, such as Paclitaxel, Sirolimus (Rapamycin), Tacrolimus, Everolimus and Zotarolimus or other cell proliferation drug in which the balloon capable of delivering sufficient, in vivo, ultrasonic energy for actively releasing the bioactive medicament within a stenosis of coronary arteries or other blood vessels in the human body. The present invention angioplasty balloon catheter may be excited to deliver ultrasonic energy in either the deflated or inflated state, or any state of partial inflation with a contrast fluid. Further, the present invention angioplasty balloon can also include one or more polymeric or other coatings below, over, or within the bioactive medicament.

The catheter has one lumen through which a guide wire may be passed for guiding the catheter to the site of stenosis. The catheter has another lumen for providing the pneumatic means or liquid contrast media to inflate and deflate the distal located angioplasty balloon. The catheter may have another lumen for the wires to electrically connect the proximal ultrasonic means to the distal ultrasonic means, although the wires can be included within the guide wire lumen, the inflation/deflation lumen, or integrated within the polymeric materials of the catheter.

The polymer coating is designed to remain intact during transport of the balloon catheter through the bloodstream until ultrasonic energy, estimated to range from 20,000 Hz to 10 MHz and with a preferable range from 200,000 to 1 MHz and less than 10 watts to cause it to break up and release the medicament(s).

According to the method of the invention, the catheter is guided to the site of arterial stenosis by means of a guiding catheter and a flexible guide wire. When the catheter has crossed the stenosis, the balloon may be inflated and the ultrasonic energy is initiated. The inflation of the balloon causes the vibrating surface of the balloon and the medicament/bioactive coating to remain in mechanical contact of the artery tissues and actively release the medicaments within the tissues. The ultrasound energy, and its driving parameters, optimally configured to drive medication into tissue while avoiding unwanted tissue heating and damage. Repeated inflations and deflations of the balloon allow for flushing of the ablated material by perfusion of blood.

Ultrasound energy has several unique properties that provide advantages in delivering energy to perform a function in the heart. First, ultrasound does not interact with blood. It passes through blood until it contacts tissue where it is absorbed. This property enables the energy to be used without heating blood that can lead to thrombus formation, a very important consideration in any procedure in the coronary arteries. This property is best exemplified in ultrasound imaging, where the blood is dark (lack of absorption) due to a lack of interaction and tissue is bright (ultrasound energy is absorbed). Second, ultrasound has a long established history of safe use in the human body. It is the preferred modality for imaging human fetus during pregnancy. Third, there is a growing body of scientific research suggest that ultrasound energy may be utilized to help drive medication into targeted areas of the body. In cancer research for example, local drug delivery is enhanced by the use of ultrasound energy that is intended to promote localized absorption of the drug into a specified targeted area.

### BRIEF DESCRIPTION OF THE DRAWINGS

FIG. 1 is a perspective view of the device of the present invention shown operationally positioned within a patient for the infusion of medication into a vessel wall;

FIG. 2 is a perspective view of a portion of an artery of a patient showing the intima, media and adventitia layers;

FIG. 3 is a perspective view of a portion of an artery of a patient showing a circumferential dispersement of a medicament (in phantom) in accordance with the method of the present invention;

FIG. 4a is a cross-sectional view of the one embodiment of the present invention in its intended environment as used in cooperation with an inflatable angioplasty balloon.

FIG. 4b is a cross-sectional view of another embodiment of the present invention in its intended environment as used in cooperation with an inflatable angioplasty balloon.

FIG. 5 is a perspective view of the device of the present invention catheter and active medicament delivery angioplasty balloon;

FIG. 6a is a cross-sectional view of the proximal section of the present invention catheter in combination with an active releasing medicament angioplasty balloon, as seen along line 6a-6a in FIG. 5;

FIG. 6b is a cross-sectional view of the distal section of the present invention catheter in combination with an active delivery medicament angioplasty balloon, as seen along line 6b-6b in FIG. 5;

FIG. 7 is a cross-sectional view of the first embodiment of the distal expandable angioplasty balloon having a medicament coating and showing the ultrasonic transducer located in the proximal end of the balloon.

FIG. 7a is a cross-section view of the active delivery medicament angioplasty balloon, as seen along line 7a-7a in FIG. 7.

FIG. 8 is a cross-sectional view of the second embodiment of the distal expandable angioplasty balloon including cutting edges showing the ultrasonic transducer located in the proximal end of the balloon.

FIG. 8a is a cross-section view of the active delivery medicament angioplasty balloon with cutting edges, as seen along line 8a-8a in FIG. 8.

FIG. 9 is a cross-sectional view of the second embodiment of the distal expandable angioplasty balloon with cutting edges having a medicament coating and showing the ultrasonic transducer located in the proximal end of the balloon.

FIG. 9a is a cross-section view of the active delivery medicament angioplasty balloon with cutting edges, as seen along line 9a-9a in FIG. 9.

FIG. 10 is a perspective view of the present invention angioplasty balloon showing the deployment of the medicament(s) from the balloon when ultrasonic energy is applied.

FIG. 11 is perspective view of the present invention active delivery medicament angioplasty balloon showing the medicament sandwiched between two non-medicament layers.

FIG. 12 is a partial cross-sectional view of the first embodiment of the distal expandable angioplasty balloon in a deflated configuration used to deliver a stent with a medicament coating and showing the ultrasonic transducer located in the proximal end of the balloon.

FIG. 13 is a partial cross-sectional view of the first embodiment of the distal expandable angioplasty balloon used to deliver a stent with a medicament coating that has been expanded within an artery and showing the ultrasonic transducer located in the proximal end of the balloon.

FIG. 14 is a partial cross-sectional view of the first embodiment of the distal expandable angioplasty balloon that was used to deliver a stent with a medicament coating that has been deflated configuration after delivering the stent within an artery and showing the ultrasonic transducer located in the proximal end of the balloon.

FIG. 15 is a perspective view of the device of the present invention showing the catheter and active medicament delivery angioplasty balloon with the ultrasonic generator and inflation/deflation device attached to the proximal end of the catheter;

FIG. 16 is a cross-sectional view of the ultrasonic transducer that is located in the angioplasty balloon.

### DESCRIPTION OF THE PREFERRED EMBODIMENTS

Generally, the catheter of the present invention comprises a multi-lumen tubular member having at least two lumens. The first lumen is used to accommodate a flexible guide wire and the second lumen forms a fluid path for a contrast fluid which serves to inflate or deflate the angioplasty balloon located at the distal end of the catheter (or a fixed guide wire design). A pair of wires may be in a third lumen, incorporated into one of the other lumens, or integrated within the catheter material whereby the wires provide electrical communication between the ultrasonic generator and the transducer.

The present invention is angioplasty balloon catheter which is coated with a bioactive medicament, such as Paclitaxel, Sirolimus (Rapamycin), Tacrolimus, Everolimus and Zotarolimus or other cell proliferation drug in which the balloon capable of delivering sufficient, in vivo, ultrasonic energy for actively releasing the bioactive medicament within a stenosis of coronary arteries or other blood vessels in the human body. The present invention angioplasty balloon catheter may be excited to deliver ultrasonic energy in either the deflated or inflated state, or any state of partial inflation with a contrast fluid. Further, the present invention angioplasty balloon can also include one or more polymeric or other coatings below, over, or within the bioactive medicament.

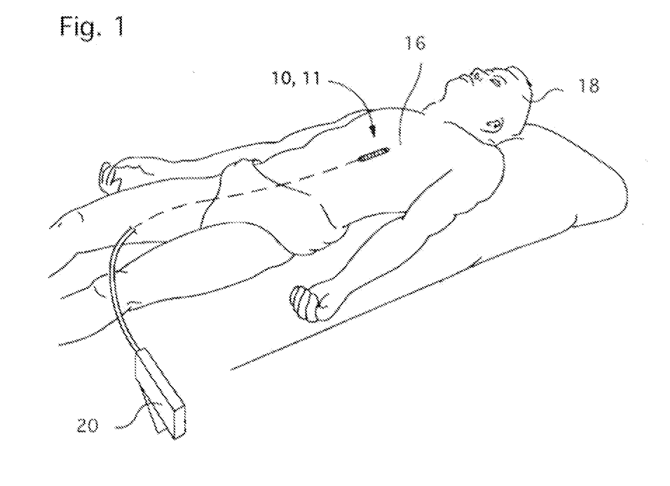

Referring initially to FIG. 1, a balloon device 10 for actively delivering a medicament 12 into a wall 14 of a living blood vessel 16 in accordance with the method of the present invention is shown positioned in an upper body, blood vessel 16 of a patient 18. It is to be appreciated that the present method can be used in arteries and other vessels throughout the body of the patient 18. A connector 20 is located on the proximal end of the catheter for engaging the ultrasonic generation means and the inflation/deflation means.

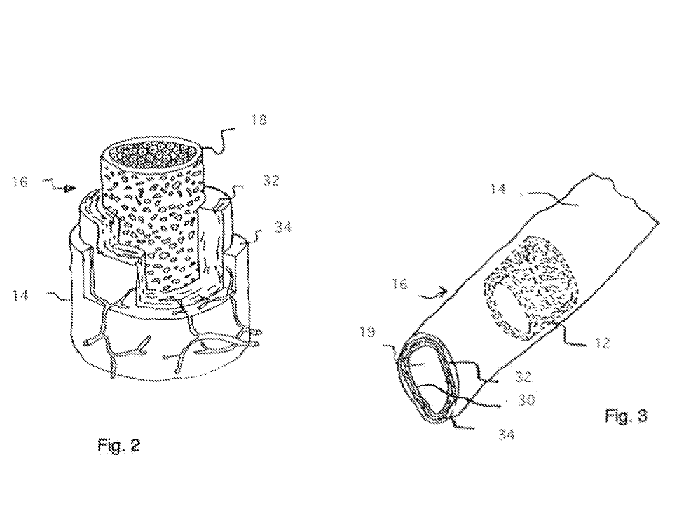

FIG. 2 shows the wall 14 of an arterial blood vessel 16 having three layers of importance for the present invention, the intima 30, the media 32 and the adventitia 34.

As shown in FIG. 3, the intima 30 surrounds the lumen 19 of the blood vessel 16. Importantly, as provided in detail below, the device 10 when used in accordance with the method provided herein, allows for a substantially circumferential dispersion of the medicament 12 within the wall 14 of the blood vessel 16. Further, in accordance with the present method, a circumferential dispersion of the medicament 10 can be made within one of the layers 30, 32, 34 of wall 16 of the blood vessel 16.

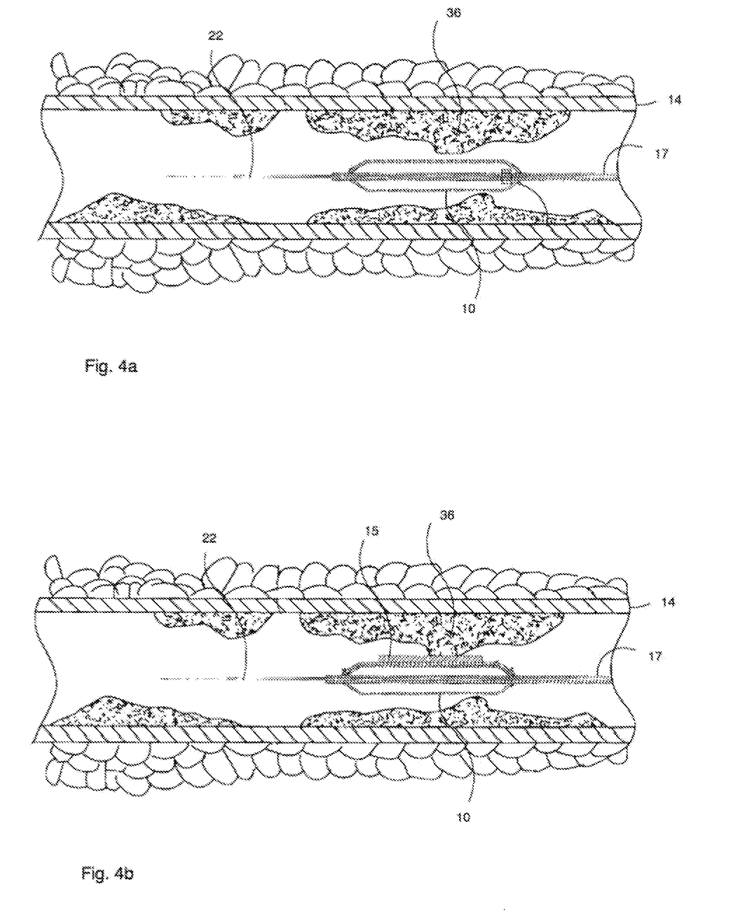

Referring now to FIG. 4b, the first embodiment of the active medicament delivery balloon 11 is shown being used in its intended environment. More specifically, the active medicament delivery angioplasty balloon 11 has been inserted into a vessel 14 and advanced along the guide wire 22 for positioning across a stenosis 36. Upon proper positioning, the active medicament delivery angioplasty balloon 10 is inflated causing outside balloon surface to come in physical contact stenosis 36 within the vessel wall 14. Upon expansion the angioplasty balloon 34 dilates the vessel 58. Simultaneously or just subsequently, one or more medicaments 12 which are coated on the angioplasty balloon 10 are actively delivered into the vessel wall 14 upon initiating the ultrasonic energy. Following dilation of the vessel and delivery of one or more medicaments, the angioplasty balloon 10 is deflated, balloon 11 to return to its original shape. The combination of the angioplasty balloon 10 and guide wire 22 is then removed from the vessel wall 14 and patient 18.

Referring now to FIG. 4b, the second embodiment of the active medicament delivery balloon 11 has one or more cutting edges 15 is shown being used in its intended environment. More specifically, the one of more cutting edges 15 are shown mounted on the active medicament delivery angioplasty balloon 11, with the combination having been inserted into a vessel 14 and advanced along the guide wire 22 for positioning across a stenosis 36. Upon proper positioning, the active medicament delivery angioplasty balloon 11 is inflated causing the cutting edges 15, to move outwardly in a radial direction. As the cutting edge 15 moves radially outward, it creates longitudinal incisions in the stenosis 36 allowing the angioplasty balloon 34 to dilate the vessel 58. Simultaneously or just subsequently, one or more medicaments 12 which are coated on the angioplasty balloon 11 and alternately, also on the cutting edges 15, are actively delivered into the vessel wall 14 upon initiating the ultrasonic energy. Following dilation of the vessel and delivery of one or more medicaments, the angioplasty balloon 11 is deflated, causing the cutting edges 15 and balloon 11 to return to its original shape. The combination of the angioplasty balloon 11, and cutting edges 15, and the guide wire 22, is then removed from the vessel wall 14 and patient 18.

The guide wire 22 may be made of a suitable material such as stainless steel or other metallic materials. Any number of conventionally available guide wires may be chosen for insertion into the guide wire lumen. It is anticipated by the Applicants that a fixed wire design can be utilized with the present invention.

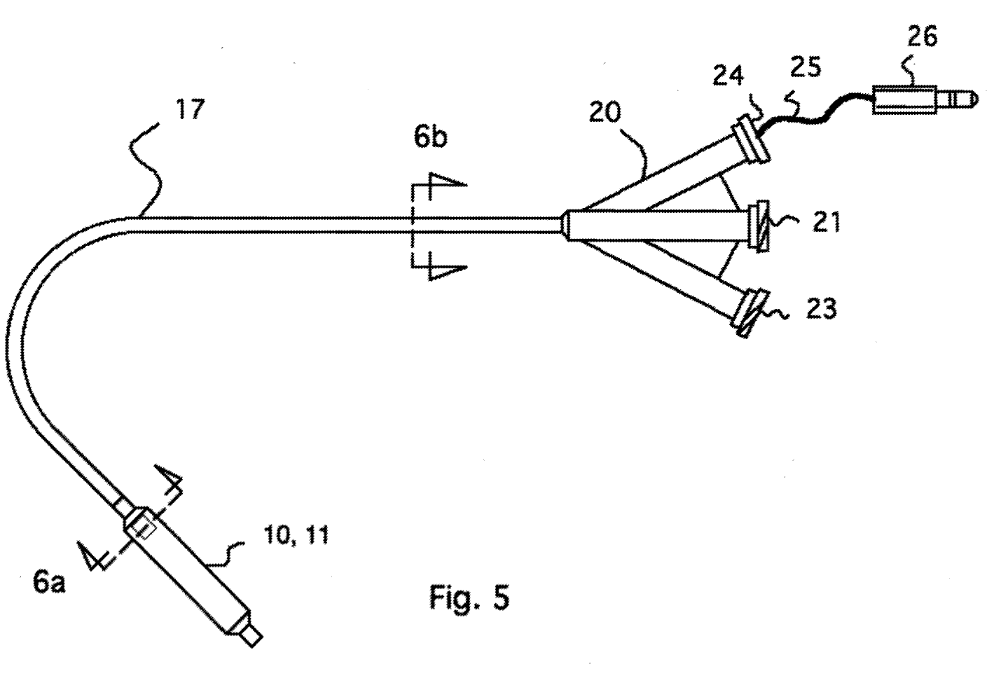

Turning now to FIG. 5, the proximal end of the present invention catheter is shown having a three port connector 20. A first port 25 is shown with a connecting jack 26 that is used to connect with an ultrasonic generator. The connecting jack 26 is electrically engaged to a pair of wires 25 which travel through a first lumen in the catheter body to the distal end where the pair of wires 25 terminate at the piezoelectric transducer 27 proving electrical conductivity to provide for the application of an ultrasonic energy to the piezoelectric transducer 27 in the balloon at the distal end of the catheter. A second port 21 continues as a second lumen throughout the longitudinal length of the catheter and exits distally from the expandable balloon. The second port and associated lumen is designed to receive a standard guidewire 22 for facilitating advancement of the balloon catheter through the tortuous vasculature of a patient's arterial system. A first section has a inside diameter which can snugly accommodate bonding (adhesive, heat or other bonding technology) of inner tube. The third port 23 is connected to an inflation/deflation lumen 23 that is in coaxial association with the outside surface 17 of the catheter body. The inflation/deflation lumen is in pneumatic or fluid communication with the distally located balloon and is designed to provide a means to inflate and deflate the balloon. The connector 20 may be formed of polycarbonate, Lexan, or any other polymeric material having resilience, non-conductivity, and strength qualities. The inner proximal end of the catheter 17 is attached by adhesive or heat bonding to the inner surface three port connector 20. The polymeric materials of these surfaces which are joined together preferably are treated to enhance bonding according to a method which does not change the inherent properties of the materials.

Also shown in FIG. 5., located at the distal end of the catheter 17 is the expandable balloon 10, 11 with the piezoelectric transducer 27 located within the proximal end of the balloon.

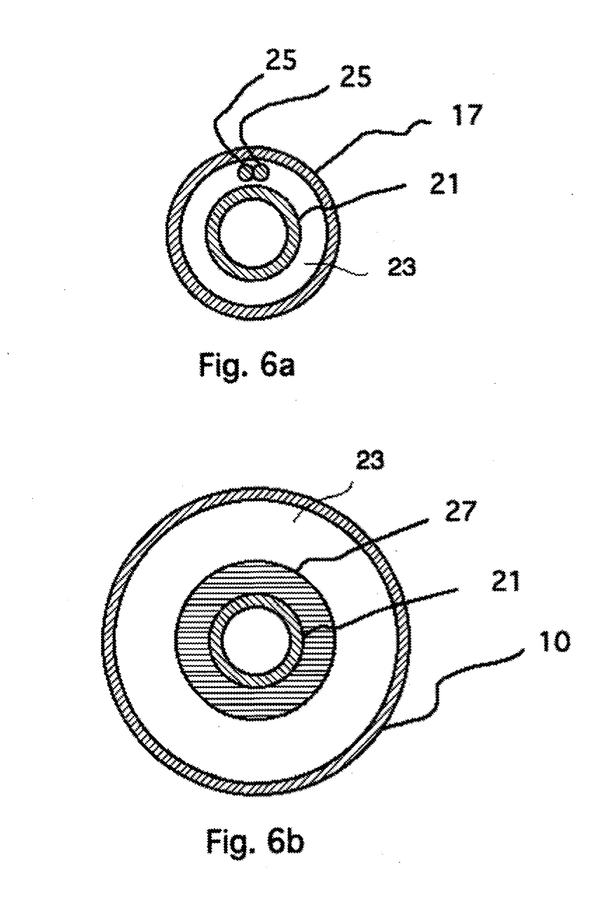

FIG. 6a is a cross-sectional view of the proximal section of the present invention catheter in combination with an active releasing medicament angioplasty balloon, as seen along line 6a-6a in FIG. 5 showing the relative position of the pair of ultrasonic transfer wires 25, the guide wire tubular member 21 (with guide wire lumen) in coaxial relationship with the outer catheter body 17, and the inflation/deflation lumen 23 located between the guide wire tubular member and the outer catheter body 17. The pair of ultrasonic transfer wires that extend from the connecting jack 26 along the longitudinal length of catheter and terminate at the piezoelectric transducer 27. The guide wire tubular member 21 extends from the second port 21 located at the proximal end of the catheter traveling along the longitudinal length of the catheter and finally exiting from the distal end of the balloon 10, 11. The inflation/deflation lumen 23 extends third port 23 along the longitudinal length of the catheter and pneumatically or fluidly connected to the distal balloon 10, 11.

FIG. 6b is a cross-sectional view of the distal section of the present invention catheter in combination with an active delivery medicament angioplasty balloon, as seen along line 6b-6b in FIG. 5 showing the relative position of the guide wire tubular member 21 h is in coaxial relationship with a substantially tubular shaped (shown as circular in this FIG.) piezoelectric transducer 27, and whereby both the guide wire tubular member 21 and the piezoelectric transducer 27 are in coaxial relationship with the outer catheter body 17. The lumen of the guide wire tubular member 21 should preferably be in the range from 0.010 to 0.020 inches to facilitate free passage of a typical guide wire having a diameter of between 0.014 and 0.018 inches. The wall thickness of the guide wire tubular member 21 should be sufficient to withstand the pressure of the fluid applied in the inflation/deflation pathway 23 for balloon inflation or deflation, which will typically be in the range of 2 to 16 atmospheres. In addition, the catheter body 17 should have an outer diameter slightly less than that of the proximal shoulder of the balloon to facilitate bonding (adhesive, heat or other bonding technology) of the balloon. Balloon and outer tube dimensions should be selected such that the outer diameter of the catheter body 17 is in the range of 0.020″ to 0.060″, with a preferred range of 0.025″ to 0.050″ inches to facilitate steering and advancing the catheter through sharp and torturous turns without exerting undue transverse pressure on the guide wire which would result in resistance to advancement of the balloon toward the stenosis site. Such flexibility is required to access certain arterial segments coronary and peripheral arteries.

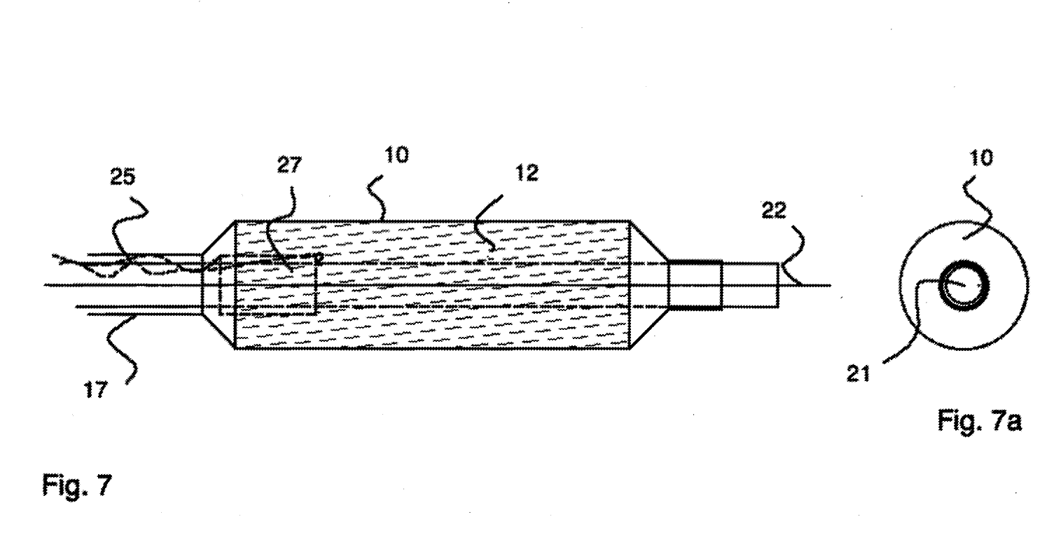

FIG. 7 is a cross-sectional view of the first embodiment of the distal expandable angioplasty balloon 10 having a medicament coating 12 substantially layered or coated on the balloon. It is anticipated by the Applicants that more than one layers of more than one medicament can be utilized with the present invention. Also shown is the proximal position of the piezoelectric transducer 27 electrically connected to the pair of ultrasonic transfer wires 25. A guide wire 22 extends beyond the distal end of the balloon 10.

FIG. 7a is a cross-section view of the active delivery medicament angioplasty balloon 10, as seen along line 7a-7a in FIG. 7. Shown in FIG. 7a is the guide wire 21 located in a lumen within the present invention active delivery balloon 10.

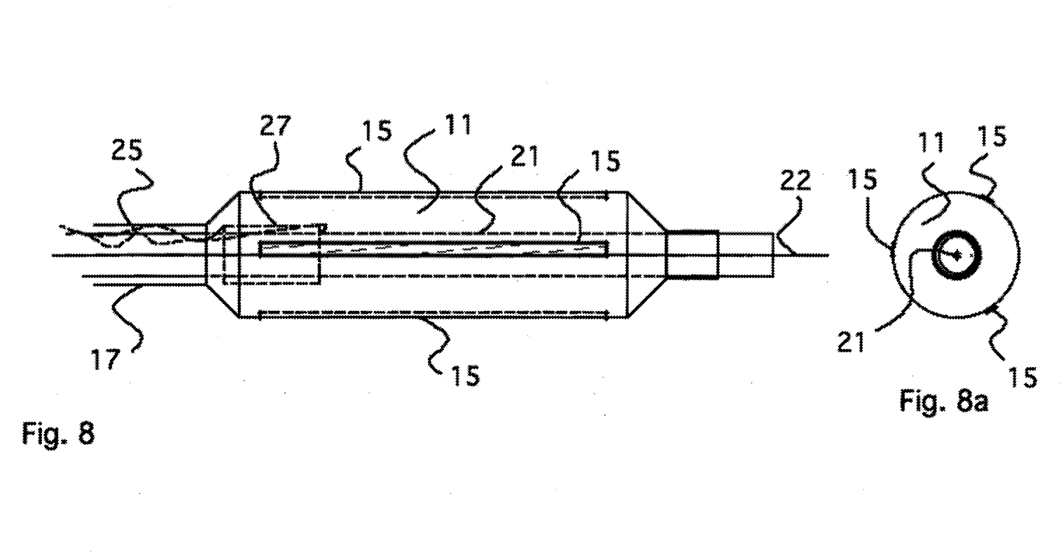

FIG. 8 is a cross-sectional view of the second embodiment of the distal expandable angioplasty balloon 11 having one or more cutting members 15 attached to the surface of the balloon 11. Also shown is the proximal position of the piezoelectric transducer 27 electrically connected to the pair of ultrasonic transfer wires 25. A guide wire 22 extends beyond the distal end of the balloon 10.

FIG. 8a is a cross-section view of the active delivery medicament angioplasty balloon 11 with cutting edges 15, as seen along line 8a-8a in FIG. 8. Shown in FIG. 8a is the guide wire 21 located in a lumen within the present invention active delivery balloon 11.

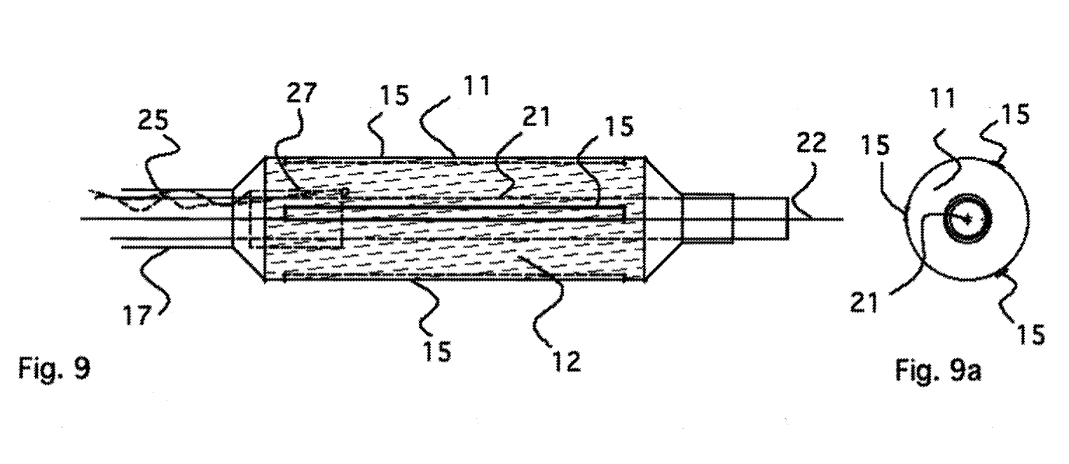

FIG. 9 is another cross-sectional view of the second embodiment of the distal expandable angioplasty balloon 11 having one or more cutting edges 15 attached to the surface of the balloon 11. A coating 12 that substantially layers or coats the balloon is shown. It is anticipated by the Applicants that more than one layers of more than one medicament can be utilized with the present invention. It is also anticipated by the Applicants that the medicament may be coated only the balloon 11, only on the cutting edges 15, or on both the balloon 11 and the cutting edges 15. Furthermore, it is anticipated by the Applicants that one or more layers of medicaments layered or coated on the balloon 11 may be different that the one or more layers or coatings of medicaments on the cutting edges 15. Also shown is the proximal position of the piezoelectric transducer 27 electrically connected to the pair of ultrasonic transfer wires 25. A guide wire 22 extends beyond the distal end of the balloon 10.

FIG. 9a is a cross-section view of the active delivery medicament angioplasty balloon 11 with cutting edges 15, as seen along line 9a-9a in FIG. 9. Shown in FIG. 9a is the guide wire 22 located in a lumen within the present invention active delivery balloon 11.

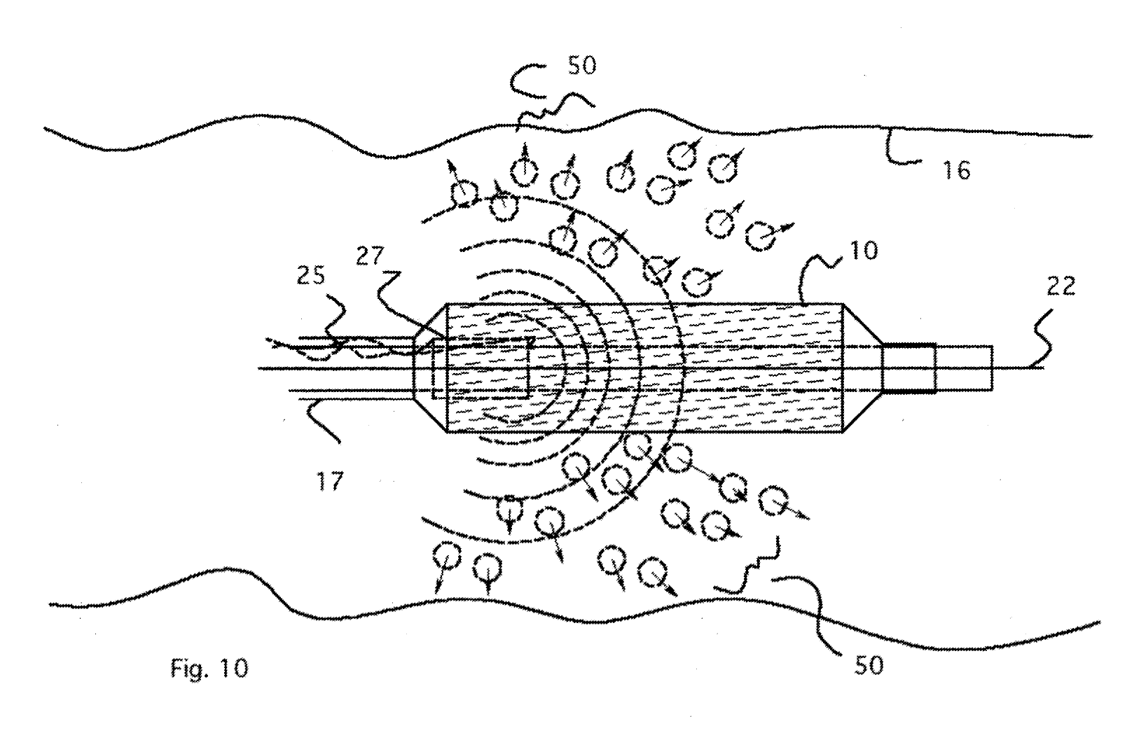

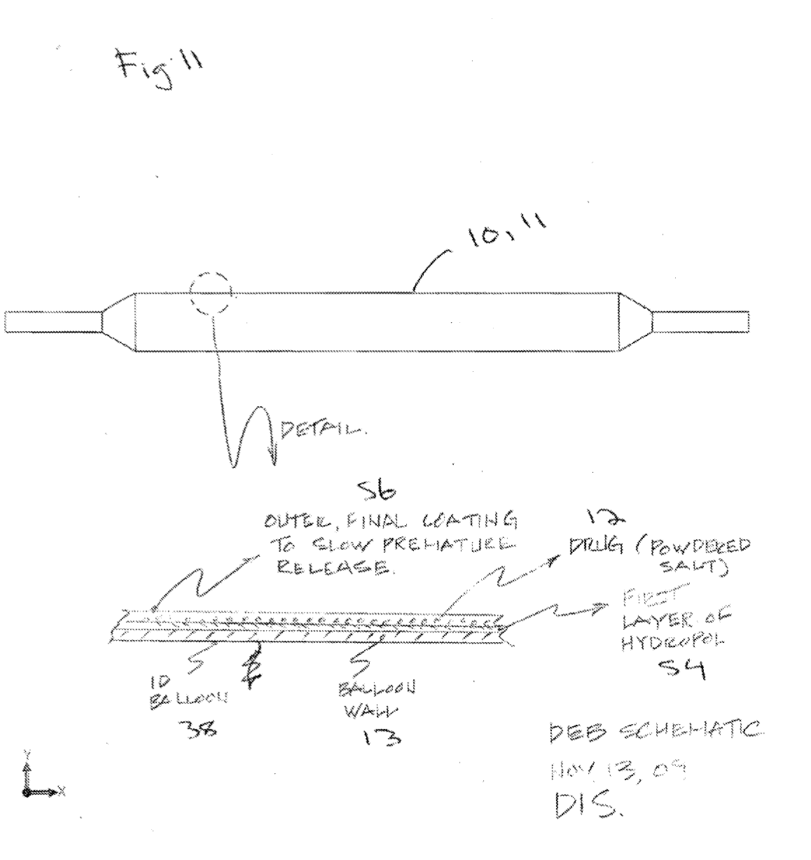

FIG. 10 is a perspective view of the present invention angioplasty balloon 10, 11 showing the active deployment of the medicament(s) 50 from the balloon when ultrasonic energy 52 is applied. The ultrasonic energy is generated from the piezoelectric transducer 27 which is electrically connected to ultrasonic transfer wires 25. Also shown is the guide wire 22 located in a lumen within the present invention active delivery balloon 10, 11.

FIG. 11 is perspective view of the present invention active delivery medicament angioplasty balloon 10, 11 showing the medicament sandwiched between two non-medicament layers 54 and 56. The innermost layer 54 can consist of a hydrophilic (hydrogel) material. The hydrophilic material will function to removably engage the medicament on the surface of the balloon. The outer layer 56 function to resist pre-mature releasing of the medicament during the advancing step of the interventional procedure.

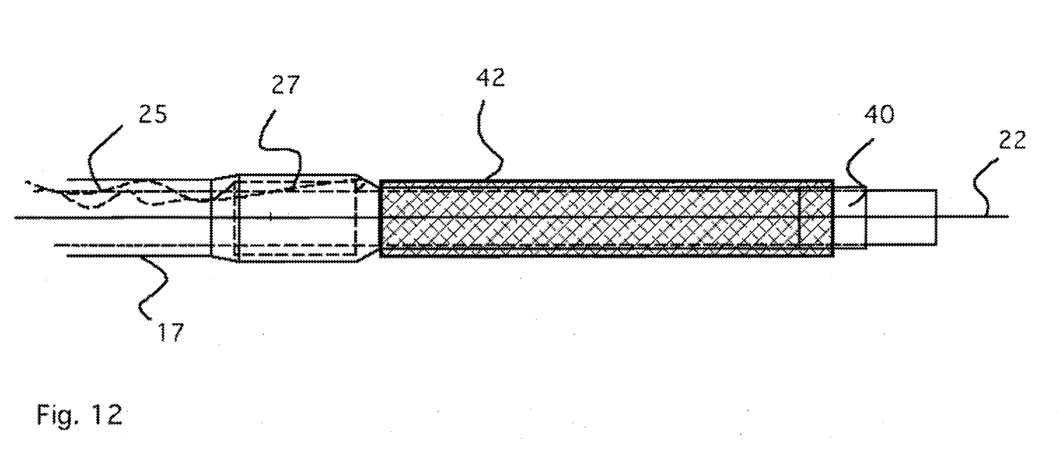

FIG. 12 is a partial cross-sectional view of the first embodiment of the distal expandable active medicament delivery balloon in a deflated configuration 40 used to deliver a stent 42 with a medicament coating and showing the ultrasonic transducer located in the proximal end of the balloon. It is anticipated by the Applicants that the medicament may be coated only the balloon 40, only on the stent 42, or on both the balloon 40 and the stent 42. Also shown in is the guide wire 22 located in a lumen within the present invention active delivery balloon 10.

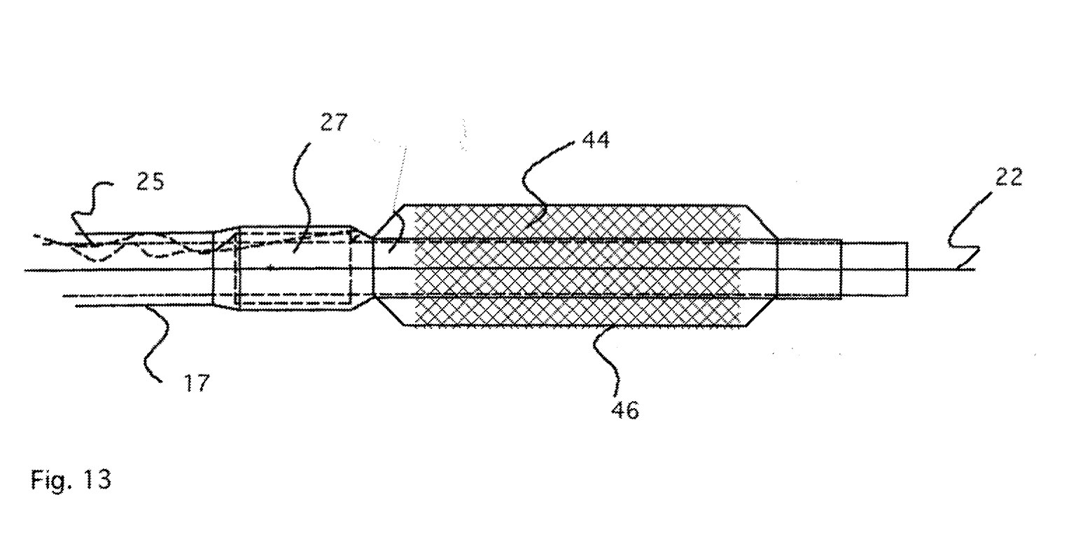

FIG. 13 is a partial cross-sectional view of the first embodiment of the distal expandable active medicament delivery balloon in an expanded configuration 44 used to deliver and expand a stent 46 with a medicament coating that has been expanded within an artery. Located on the proximal end of the expanding balloon 44 is the ultrasonic piezoelectric transducer 17 electrically connected to a pair of ultrasonic transfer wires 25. Also shown is the guide wire 22 located in a lumen within the present invention active delivery balloon 44.

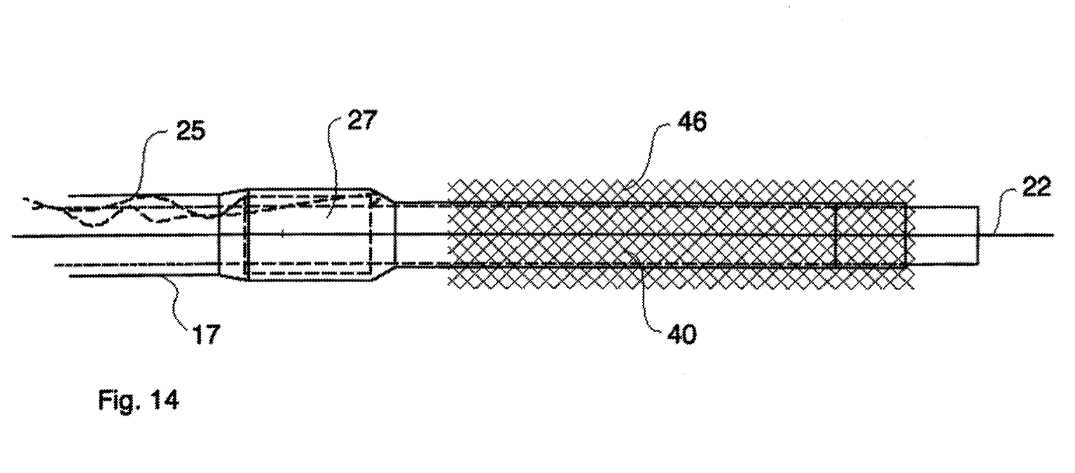

FIG. 14 is a partial cross-sectional view of the first embodiment of the distal expandable angioplasty balloon that was used to deliver a stent with a medicament coating that is a post delivery deflated configuration 40 after delivering the expanded stent 46 within an artery and showing the ultrasonic transducer located in the proximal end of the balloon. Located on the proximal end of the deflated configuration balloon 46 is the ultrasonic piezoelectric transducer 17 electrically connected to a pair of ultrasonic transfer wires 25. Also shown is the guide wire 22 located in a lumen within the present invention active delivery balloon 40.

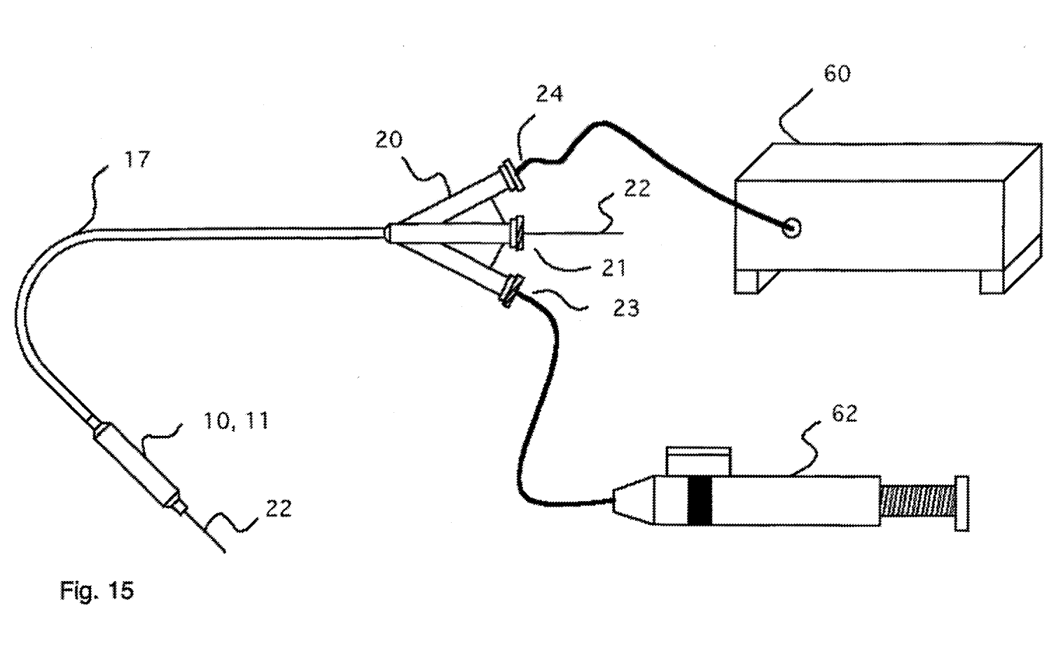

FIG. 15 is a perspective view of the device of the present invention showing the catheter 17 and active medicament delivery balloon 10, 11 with the ultrasonic generator 60 connected to a piezoelectric transducer (not shown in this FIG) by ultrasonic wires through port 24 of three port connector 20. An inflation/deflation device 62 is attached to three port connector 20 at inflation/deflation port 23. Also shown is the guide wire 22 entering guide wire port 21. The length of the catheter should be approximately 135 cm, as is typical for conventional percutaneous transluminal coronary angioplasty catheters. The catheter may be marked at a regular interval, 10 cm by way of example, with a radio-opaque marker, as is typically done with conventional catheters known in the art. It is preferable to use frequencies in the range of about 10 kHz to about 40 kHz but other values can be utilized. In clinical use, the power output of the transducer should not exceed 25 watts to prevent necrosis of the endothelial, medial or adventitia cells may occur. The ultrasonic generator 60 used should have a fixed output frequency and a maximum amplitude setting to avoid misuse or inadvertent injury to the patient. The active medicament delivery balloon 10, 11, 40 and 44 may be driven, by way of example, with a 20 kHz signal with a range of 5 to 25-volt RMS amplitude on a 50% duty cycle of 30 milliseconds at 60 second intervals. In other words, such a drive would deliver 600 cycles of 25 RMS volts Max. for 30 milliseconds, then rest for 30 milliseconds, and this can be done 1000 times for every 60 second interval, after which the drive may be stopped to evaluate progress. The balloon may be driven by any waveform, such as a square wave or a sinusoidal wave, or by pulsed d.c.

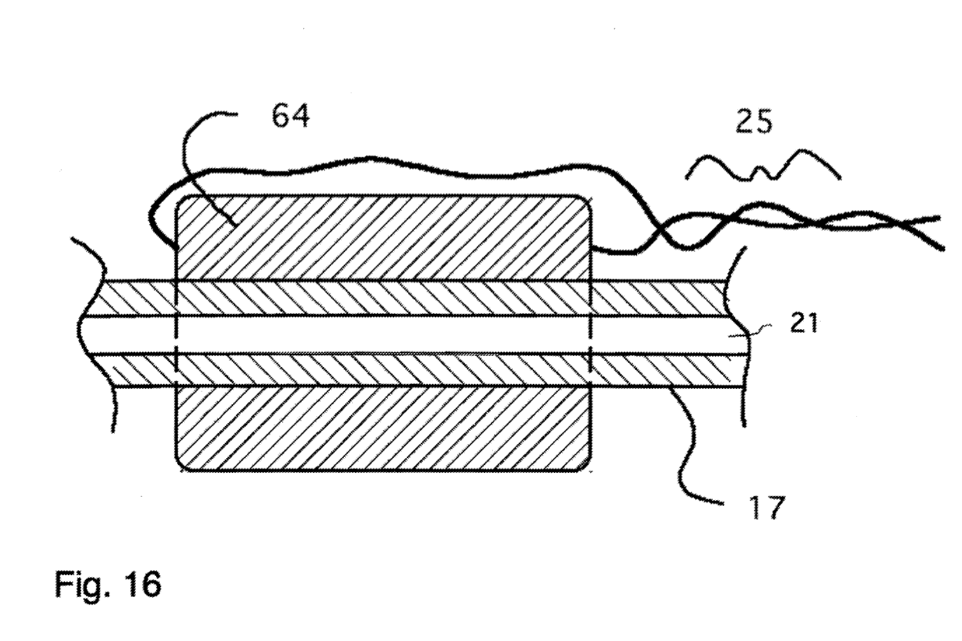

FIG. 16 is a cross-sectional view of the ultrasonic piezoelectric transducer 17 that is located in a proximal location with the angioplasty balloon 10, 11, 40 and 44 and surrounds the catheter shaft 17.

Provided below are some experiments used to support the present invention.

Balloon Material:

Experiment 1

PA—Polyamide, Nylon

PEBA—Pebax Nylon ether block copolymer

PET

PE

PU—Polyurethane

Hytrel

This experiment was performed just to get feel for the polymer as starting point and to learn.

### Material Used:

1. Acetone
2. Kimax 25 ml volumetric flask
3. Kimax 10 ml volumetric flask
4. 5 cc syringe
5. Mace Hydropol 6B—This is a one part moisture curing Polyurethane prepolymer (low weight polymer between monomer and polymer) that absorbs water 500× its own weight.
6. 2.5 mm PA12 balloons
7. 1/16^{th} RNF 100 shrink tubing

### Procedure:

1. Mix acetone and hydropol. Two mixture was made: 25 acetone to 1 part by volume (4%) and 10 acetone to 1 part by volume (10%).
2. Seal the end of the balloon tubing with shrink tubing and heat gun set at 500 deg F.
3. Dip balloons in 4% solution and place on the foam rest. Repeat for 5 balloons total.
4. Dip balloons in 10% solution and place on the foam rest. Repeat for 4 balloons total.
5. Set aside and allow to cure.

### Visual:

Before leaving the lab, performed a quick visual inspection of the dipped balloons. The 4% solution dipped balloons had very low coverage. It looked as if the prepol was a net pattern with wet and un-wet spot forming the net pattern. The 10% solution formed a much better coverage and looked like about 80% coverage or better.

### Experiment 2

This was the first experience with the coated balloons. The adhesion properties were looked at as wall thickness of the coated balloons was measured with a blade micrometer and performed another visual.
The uncoated balloon thickness was 0.0012 inch.
4% solution coated—0.0012, 0.0012, 0.0012
10% solution coated—0.0019, 0.0015, 0.0017, and 0.0015.
The 10% solution coated a much thicker coating with much better coverage with about 80% or better coverage. With proper dipping process this can improve.
The dipped balloons were soaked in water to see if the coating would help reduce friction but it did not feel anymore slippery that the raw balloons. It actually felt stickier.
The adhesion of the coating on the balloons looked very good. Did not scrape off.
Placed some prepol on LDPE sheet and mixed it water. This caused the prepol to bubble and foam up. The bubbles were large and as soon the bubbles became big enough and rose to surface, it popped. After a while, as the material started to cure, the mixture started to become a closed cell foam.
In a solution, sprinkle with water and mixing did not cause it to bubble.

In order to mimic drug delivery situation, balloons were coated first with 10% solution and then powdered salt was rolled on to the balloon.

### Material:

1. Table salt—powdered using roller
2. Aluminum plate for powder the salt
3. Acetone
4. Kimax 25 ml volumetric flask
5. Kimax 10 ml volumetric flask
6. 5 cc syringe
7. Mace Hydropol 6B—This is a one part moisture curing Polyurethane prepolymer (low weight polymer between monomer and polymer) that absorbs water 500× its own weight.
8. 2.5 mm PA12 balloons
9. 1/16^{th} RNF 100 shrink tubing

### Procedure:

1. For the solution, used the same one mixed previously on Oct. 19^{th}, 2009.
2. Close of the distal ends of the balloons with RNF 100.
3. Insert 0.013 PTFE coated mandrel from the proximal end of the balloon.
4. Dip in the 10% solution and place on the foam rest. Make total of 5 samples.
5. Roll in the powdered salt. Initially tried to sprinkle but it was not as effective and rolling on a small mound of salt. Repeat for all 5 samples.
6. First dipped 6 more in 10% solution
7. 2^{nd} dip in the 4% solution. This turn out to be too thin and most of salt fell off during the redipping process. Two samples were dipped in 4% solution
8. Left 3 without 2nd coating to use as control.
9. 2^{nd} dip two of these after a little wait in 10% solution.
10% solution seems to better in terms of loosing salt but it was still losing some.
10. Total samples were:
   a. 2 samples in 10%, salt, 4%
   b. 2 samples in 10%, salt, 10%
   c. 3 samples in 10%, salt, no second coat.
   d. 4 samples in 10%, salt to be 2^{nd} dipped day after to allow the first coating to cure.
11. This is a continuation of the work from the previous day.
   The remaining 4 samples were dipped in 10% solution and set aside to cure. These samples were marked to 4 stripes on the neck of the balloon.

### Experiment 3

### Electric Conductivity Measurement

The experiment was conducted to test the effectiveness of the coated balloons. The method used was to measure the electrical conductivity of the water in a beaker.

### Materials:

### 1. Tektronix Multimeter ET332

### 2. Probes

3. 150 ml beaker
4. 150 ml flask
5. Distilled water

### 6. Timer

7. Ultrasonic fogger—Alpine Corporation FG100

### Procedure:

1. Rinse out the beaker and the flask with tap water
2. Measure 150 ml of distilled water in the flask and pour into the beaker.
3. Tape the multimeter probes so the spacing remains constant and place it in the beaker allowing the tips to touch the bottom and leaned on the rim
4. Turn on the multimeter and set it the Mega Ohm setting
5. Insert the wire mandrel in the balloon.
6. Variation in testing.
7. Turn on the timer to 40 secs and insert the sample balloon. Stir the balloon so the slat would dissolve faster but no Ultrasonic fogger.
   a. Turn on the timer to 40 secs and insert the control sample balloon. Use the ultrasonic fogger but do not stir the balloon.
7. Record the resistance after the number settles to a stable number.

### Results:

Following are the results:

| | | 10% dip, salt, | 10% dip, salt, |
|---|---|---|---|
| Test method | Control | 4% dip | 10% dip |
| 6a-Stir, no | Sample 1-375k | Sample 2-400k | |
| Ultrasonic | Ohm dropped | Ohm dropped | |
| | to 130k Ohm | to 210k Ohm | |
| 6b-No stir, | | Sample 3-440K | Sample 4-370k |
| yes Ultrasonic | | Ohm but | Ohm dropped to |
| | | ultrasonic did | 330k Ohm |
| | | not come on. | Sample 5-470k |
| | | | Ohm dropped |
| | | | to 160K Ohm at |
| | | | around 20 secs. |
| | | | The US stopped |
| | | | working at 15 |
| | | | seconds. |

2. Discussion: The results look promising. The release salt after the 2^{nd} coating seems to show potential even with this coating material. Need to make more samples and test.

### Experiment 4

### Sample Preparation:

1. Before leaving the lab, prepared 15 more 2.5 mm balloons. Dipped them in 10% solution and rolled in salt immediately to allow most salt to adhere to the coating.
2. Left these to cure overnight before 2^{nd} coating them. Make some quick visual observations prepped samples for the 2^{nd} time.
3. The powder was much finer this time.
4. Some of the powder clumped together and few balloons had clumps of powdered salt adhered to it.
5. Overall the there were finer dispersion over the balloon.

Dipped 8× samples in 10% solution and left 7 without the second coating in case we needed to change directions.

Made some observations on the 4 samples that were 2^{nd} coated the day before.
1. The adhesion of salt on the balloon was quite firm.
2. There were some large salt crystal that was stick out from the surface due to the size but it sill held.

General comments and thought for further work.
1. Fine grain is better way to go.
2. Need to try different coating material like UV adhesive thinned out to make a thin coating. Try with soft durometer and low viscosity.
3. This will allow faster sample turn around.
4. Food type of jello or gel that dissolves with water but takes a bit more time may be a candidate.

What is claimed:

1. A drug eluting device suitable for use in delivering a medicament into a stenosis in a vascular vessel, comprising;
said drug eluting device compromising a catheter having an ultrasonic electrical lumen and an inflation lumen, said catheter including an inflatable balloon located at the distal end and in pneumatic communication with said inflation lumen, said balloon fabricated at least partially from a polymeric material and having piezoelectric properties;
said inflation balloon including at least one coating comprising at least one layer, wherein said at least one layer of said at least one coating comprises an adjustable matrix composition comprising a material adapted for use with an ultrasound transducer whereby the coating minimizes the release of the medicament into the bloodstream until a transducer is activated. and a bioactive medicament;
a means of delivering ultrasonic energy to said inflatable balloon via an ultrasonic electrical excitation signal whereby the inflatable balloon can be excited into ultrasonic vibratory states capable of delivering a medicament into the stenosis in the vascular vessel.

2. The drug eluting device according to claim 1, whereby said inflation balloon is a angioplasty balloon.

3. The drug eluting device according to claim 1, whereby said inflation balloon is a balloon with cutting edges.

4. A drug eluting device according to claim 1, wherein the ultrasonic electrical excitation signal is applied across the thickness of the polymeric material of the inflatable balloon.

5. A drug eluting device according to claim 1, further comprising that the inflatable balloon includes a contiguous layer of a conductive metal extending under said at least one coating.

6. A drug eluting device according to claim 5, wherein the layer of the conductive metal is silver or gold.

7. A drug eluting device according to claim 5 further having a flexible coating of an insulating material surrounding the layer of the conductive metal.

8. A drug eluting device suitable for use in delivering a medicament into a stenosis in a vascular vessel, comprising;
said drug eluting device compromising a catheter having an ultrasonic electrical lumen and an inflation lumen, said catheter including an inflatable balloon located at the distal end and in pneumatic communication with said inflation lumen, said balloon fabricated at least partially from a polymeric material and having piezoelectric properties;
said inflation balloon surrounded by a stent which includes at least one coating comprising at least one layer, wherein said at least one layer of said at least one coating comprises an adjustable matrix composition comprising a material adapted for use with an ultrasound transducer whereby the coating minimizes the release of the medicament into the bloodstream until an activation of ultrasonic electrical excitation is activated, and a bioactive medicament;
a means of delivering ultrasonic energy to said inflatable balloon via an ultrasonic electrical excitation signal whereby the inflatable balloon can be excited into ultrasonic vibratory states capable of delivering a medicament from said stent into the stenosis in the vascular vessel.

9. A drug eluting device according to claim 8, wherein the ultrasonic electrical excitation signal is applied across the thickness of the polymeric material of the inflatable balloon and across the thickness of the surrounding stent.

10. A according to claim 8, further comprising that the inflatable balloon includes a contiguous layer of a conductive metal extending under said at least one coating.

11. A catheter according to claim 10, wherein the layer of the conductive metal forming the outer surface of the catheter is silver or gold.

12. A catheter according to claim 10 further having a flexible coating of an insulating material surrounding the layer of the conductive metal.

13. A method of delivering a medicament into a stenosis in a vascular vessel, comprising;
advancing a drug elution device, said drug eluting device compromising a catheter having an ultrasonic electrical lumen and an inflation lumen, said catheter including an inflatable balloon located at the distal end and in pneumatic communication with said inflation lumen, said balloon fabricated at least partially from a polymeric material and having piezoelectric properties, said inflation balloon including at least one coating comprising at least one layer, wherein said at least one layer of said at least one coating comprises an adjustable matrix composition comprising a sol gel material and a bioactive material, a means of delivering ultrasonic energy to said inflatable balloon via an ultrasonic electrical excitation signal whereby the inflatable balloon can be excited into ultrasonic vibratory states capable of delivering a medicament into the stenosis in the vascular vessel; and
initiating ultrasonic energy to the inflation balloon.

14. A method of delivering a medicament into a stenosis in a vascular vessel, comprising;
advancing a drug elution device, said drug eluting device compromising a catheter having an ultrasonic electrical lumen and an inflation lumen, said catheter including an inflatable balloon located at the distal end and in pneumatic communication with said inflation lumen, said balloon fabricated at least partially from a polymeric material and having piezoelectric properties, said inflation balloon surrounded by a stent which includes at least one coating comprising at least one layer, wherein said at least one layer of said at least one coating comprises an adjustable matrix composition comprising a sol gel material and a bioactive material, a means of delivering ultrasonic energy to said inflatable balloon via an ultrasonic electrical excitation signal whereby the inflatable balloon can be excited into ultrasonic vibratory states capable of delivering a medicament into the stenosis in the vascular vessel; and
initiating ultrasonic energy to the inflation balloon.
